(19) United States
(12) Patent Application Publication
(10) Pub. No.: US 2024/0257948 A1
(43) Pub. Date: Aug. 1, 2024

(54) METHOD AND SYSTEM FOR FACILITATING CONSISTENT USE OF DESCRIPTORS IN RADIOLOGY REPORTS

(71) Applicant: KONINKLIJKE PHILIPS N.V., EINDHOVEN (NL)

(72) Inventor: Sawarkar ABHIVYAKTI, SWANSEA, MA (US)

(21) Appl. No.: 18/560,944

(22) PCT Filed: May 13, 2022
(86) PCT No.: PCT/EP2022/063063

§ 371 Date: Nov. 15, 2023

Related U.S. Application Data
(60) Provisional application No. 63/190,457, filed on May 19, 2021.

(51) Int. Cl. G16H 30/40; G06F 40/205; G16H 15/00; G16H 50/20; G16H 50/70

(52) U.S. Cl. CPC G16H 30/40; G06F 40/205; G16H 15/00; G16H 50/20; G16H 50/70

(57) ABSTRACT

A system and method are provided for facilitating consistent use of descriptors describing medical images. The method includes receiving contents of a radiology report from a user via a GUI, the contents including a measurement of an abnormality and descriptors in descriptive text corresponding to the measurement; extracting the measurement and the corresponding descriptors from the contents of the radiology report using an NLP algorithm; developing a machine learning model including the measurement and the descriptors, where the machine learning model determines at least one of a behavior pattern or a practice variation of the user with respect to use of the descriptor relative to industry standards and/or reporting behavior of additional users; and developing a collective machine learning model of results of the developed machine learning model regarding the behavior patterns and/or the practice variation of the user and results of developed machine learning models regarding behavior patterns and/or practice variations of the additional users.

### BACKGROUND

Radiologists routinely generate radiology reports describing medical images of patients. A radiology report typically includes, in part, a findings section that identifies what the radiologist observed in areas of the medical image and characterizes normality or abnormality of each observation, and an impressions section that summarizes the findings, assesses the condition, provides diagnoses and recommendations going forward with regard additional testing and treatment. The radiology report should be clear and succinct, using industry standard and/or commonly used terminology from an approved lexicon. For example, a radiology report may include measurements of lesions or other abnormalities in a medical image, as well as descriptions and/or diagnoses of the lesions. To be useful, the radiology report should contain certain salient features using standardized descriptors (i.e., industry standard and/or commonly used descriptors) to disambiguate the lesions, and to identify a baseline and follow-up of the diagnosis. Without these features, the radiology report will likely be incomplete and may fail to convey the seriousness or adequacy of the diagnoses. Also, missed or inconsistent use of descriptors not only indicate undesirable variations in practice, but also may have critical implications for patient care resulting from misunderstood findings, leading to substandard treatment.

However, in preparing radiology reports, radiologists often use descriptors that are not standardized, use descriptors inconsistently in describing the same or similar abnormalities (within the same radiology report and among difference radiology reports), and/or fail to use descriptors altogether in identifying abnormalities in the medical images. Currently, there is no effective measure of the inclusion of standardized descriptors, the consistent use of descriptors in multiple reports, or missing descriptors altogether. As a result, abnormalities that are not correctly identified and described in the radiology reports may be overlooked, improperly diagnosed, and/or very difficult to track for use in long term studies or data analyses. For example, according to recommended radiology practices, an imaging exam generally should be compared with previous screening or diagnostic exams, so inconsistent use of descriptors may lead to missing trends among imaging exams, such as disease progression and treatment efficacy, for example.

Accordingly, what is needed is an automated system for measuring the use, accuracy and consistency of descriptors in radiology reports. Such an automated system may enable detection of radiologists' behavior patterns and practice variations with respect to reported measurements and standardized descriptors, will improve consistency among radiology reports and diagnostic certainty.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a simplified block diagram of a system for facilitating consistent use of descriptors by users describing medical images displayed on a display including a graphical user interface (GUI), according to a representative embodiment.

FIG. 2 is a flow diagram showing a method of facilitating consistent use of descriptors by users describing medical images displayed on a display including a GUI, according to a representative embodiment.

FIG. 3 is a flow diagram of a method of applying an NPL algorithm for extracting feature measurements and corresponding descriptors from a radiology report, according to a representative embodiment.

FIG. 4 shows a portion of an illustrative radiology report input to a natural language processing (NLP) algorithm, and a corresponding output of the NLP algorithm including extracted measurements and descriptors, according to a representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a,” “an” and “the” are intended to include both singular and plural forms, unless the context clearly dictates otherwise. Additionally, the terms “comprises,” “comprising,” and/or similar terms specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

Generally, the various embodiments described herein provide an automated system to analyze radiology reports for consistent inclusion and use of standardized descriptors, enabling detection of behavior patterns and determination of practice variations of radiologists with regard to use of measurement descriptors. The embodiments further provide machine learning models to measure the practice behavior with regard to the radiologists' decisions to include certain measurement descriptors. The results of the machine learning models are a ready reference available to the radiologists to enable changes in current or subsequent radiology reports to use standardized descriptors and to conform use of descriptors, helping diagnoses towards greater definitiveness. The machine learning model results may also be used as a training tool for the radiologists in order to increase awareness, promote conformity of descriptor use, and to improve operational and reading workflow efficiency.

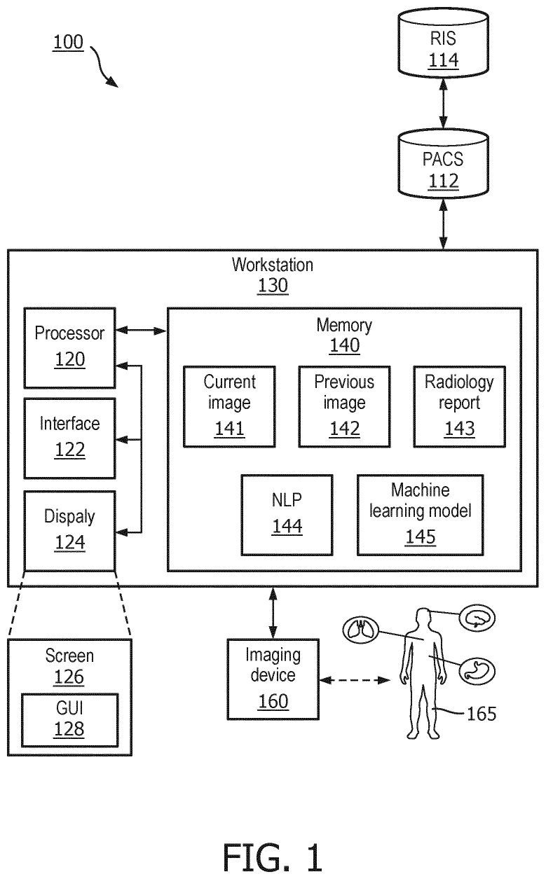

FIG. 1 is a simplified block diagram of a system for facilitating consistent use (inclusion and standardization) of descriptors by users describing medical images displayed on a display including a graphical user interface (GUI), according to a representative embodiment.

Referring to FIG. 1, system includes a workstation 130 for implementing and/or managing the processes described herein. The workstation 130 includes one or more processors indicated by processor 120, one or more memories indicated by memory 140, interface 122 and display 124. The processor 120 may interface with an imaging device 160 through an imaging interface (not shown). The imaging device 160 may be any of various types of medical imaging device/modality, including an X-ray imaging device, a computerized tomography (CT) scan device, a magnetic resonance (MR) imaging device, a positron emission tomography (PET) scan device, or an ultrasound imaging device, for example.

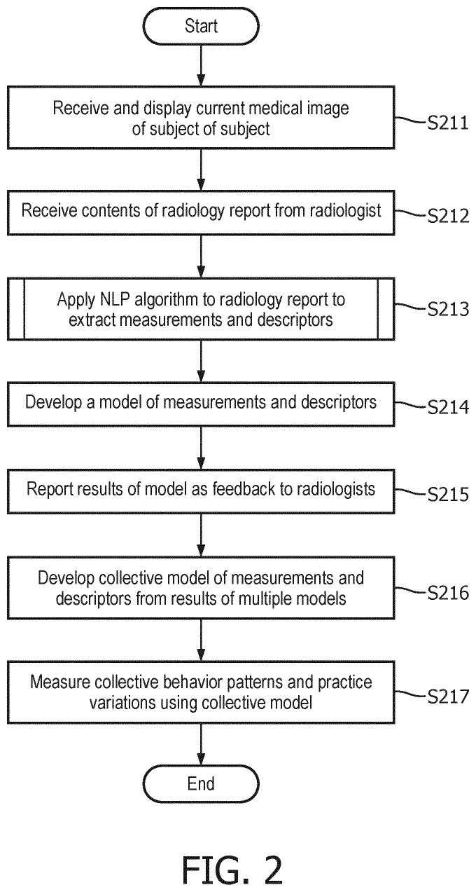

The memory 140 stores instructions executable by the processor 120. When executed, the instructions cause the processor 120 to implement one or more processes for facilitating the consistent use of descriptors by radiologists describing measured lesions in the medical images displayed on the display 124, described below with reference to FIG. 2, for example. For purposes of illustration, the memory 140 is shown to include software modules, each of which includes the instructions corresponding to an associated capability of the system 100.

The processor 120 is representative of one or more processing devices, and may be implemented by field programmable gate arrays (FPGAs), application specific integrated circuits (ASICs), a digital signal processor (DSP), a general purpose computer, a central processing unit, a computer processor, a microprocessor, a microcontroller, a state machine, programmable logic device, or combinations thereof, using any combination of hardware, software, firmware, hard-wired logic circuits, or combinations thereof. Any processing unit or processor herein may include multiple processors, parallel processors, or both. Multiple processors may be included in, or coupled to, a single device or multiple devices. The term “processor” as used herein encompasses an electronic component able to execute a program or machine executable instruction. A processor may also refer to a collection of processors within a single computer system or distributed among multiple computer systems, such as in a cloud-based or other multi-site application. Programs have software instructions performed by one or multiple processors that may be within the same computing device or which may be distributed across multiple computing devices.

The memory 140 may include main memory and/or static memory, where such memories may communicate with each other and the processor 120 via one or more buses. The memory 140 may be implemented by any number, type and combination of random access memory (RAM) and read-only memory (ROM), for example, and may store various types of information, such as software algorithms, artificial intelligence (AI) machine learning models, and computer programs, all of which are executable by the processor 120. The various types of ROM and RAM may include any number, type and combination of computer readable storage media, such as a disk drive, flash memory, an electrically programmable read-only memory (EPROM), an electrically erasable and programmable read only memory (EEPROM), registers, a hard disk, a removable disk, tape, compact disk read only memory (CD-ROM), digital versatile disk (DVD), floppy disk, Blu-ray disk, a universal serial bus (USB) drive, or any other form of storage medium known in the art. The memory 140 is a tangible storage medium for storing data and executable software instructions, and is non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The memory 140 may store software instructions and/or computer readable code that enable performance of various functions. The memory 140 may be secure and/or encrypted, or unsecure and/or unencrypted.

The system 100 also includes databases for storing information that may be used by the various software modules of the memory 140, including a picture archiving and communication systems (PACS) database 112 and a radiology information system (RIS) database 114. The databases may be implemented by any number, type and combination of RAM and ROM, for example. The various types of ROM and RAM may include any number, type and combination of computer readable storage media, such as a disk drive, flash memory, EPROM, EEPROM, registers, a hard disk, a removable disk, tape, CD-ROM, DVD, floppy disk, Blu-ray disk, USB drive, or any other form of storage medium known in the art. The databases are tangible storage mediums for storing data and executable software instructions and are non-transitory during the time data and software instructions are stored therein. The databases may be secure and/or encrypted, or unsecure and/or unencrypted. For purposes of illustration, the PACS database 112 and the RIS database 114 are shown as separate databases, although it is understood that they may be combined, and/or included in the memory 140, without departing from the scope of the present teachings.

The processor 120 may include or have access to an artificial intelligence (AI) engine, which may be implemented as software that provides artificial intelligence (e.g., NLP algorithms) and applies machine learning described herein. The AI engine may reside in any of various components in addition to or other than the processor 120, such as the memory 140, an external server, and/or the cloud, for example. When the AI engine is implemented in a cloud, such as at a data center, for example, the AI engine may be connected to the processor 120 via the internet using one or more wired and/or wireless connection(s).

The interface 122 may include a user and/or network interface for providing information and data output by the processor 120 and/or the memory 140 to the user and/or for receiving information and data input by the user. That is, the interface 122 enables the user to enter data and to control or manipulate aspects of the processes described herein, and also enables the processor 120 to indicate the effects of the user's control or manipulation. All or a portion of the interface 122 may be implemented by a graphical user interface (GUI), such as GUI 128 viewable on the display 124, discussed below. The interface 122 may include one or more of ports, disk drives, wireless antennas, or other types of receiver circuitry. The interface 122 may further connect one or more user interfaces, such as a mouse, a keyboard, a trackball, a joystick, a microphone, a video camera, a touchpad, a touchscreen, voice or gesture recognition captured by a microphone or video camera, for example.

The display 124, also referred to as a diagnostic viewer, may be a monitor such as a computer monitor, a television, a liquid crystal display (LCD), an organic light emitting diode (OLED), a flat panel display, a solid-state display, or a cathode ray tube (CRT) display, or an electronic whiteboard, for example. The display 124 includes a screen 126 for viewing internal images of a subject (patient) 165, along with various features described herein to assist the user in accurately and efficiently reading the medical images, as well as the GUI 128 to enable the user to interact with the displayed images and features. The user is able to personalize the various features of the GUI 128, discussed below, by creating specific alerts and reminders, for example.

Referring to the memory 140, current image module 141 is configured to receive (and process) current medical image corresponding to the subject 165 for display on the display 124. The current medical image is the image currently being read/interpreted by the user (e.g., radiologist) during a reading workflow. The current medical image may be received from the imaging device 160, for example, during a contemporaneous imaging session of the subject. Alternatively, the current image module 141 may retrieve the current medical image from the PACS database 112, which has been stored from the imaging session, but not yet read by the user. The current medical image is displayed on the screen 126 to enable analysis by the user for preparing a radiology report, which in includes measurements of various abnormalities (e.g., lesions, tumors) identified in the current medical image and corresponding descriptive text.

The memory 140 may optionally include previous image module 142, which receives previous medical image(s) of the subject 165 from the PACS database 112. All or part of the previous medical image may be displayed, jointly or separately, with the current medical image on the screen 126 to enable visual comparison by the user. When displayed jointly, the previous and current medical images may be registered with one another.

Previous radiology report module 143 is configured to retrieve a previous radiology report from the PACS database 112 and/or the RIS database 114 regarding the subject 165. The radiology report provides analysis and findings of previous imaging of the subject 165, and may correspond to a previous medical image retrieved by the previous image module 142. The radiology report includes information about the subject 165, details on the previous imaging session, and measurements and medical descriptive text entered by the user who viewed and analyzed the previous medical image associated with the radiology report. Relevant portions of the radiology report may be displayed on the display 124 in order to emphasize information to the user that may be helpful in analyzing the current medical image, such as past measurements of the same abnormalities viewed in the current medical image.

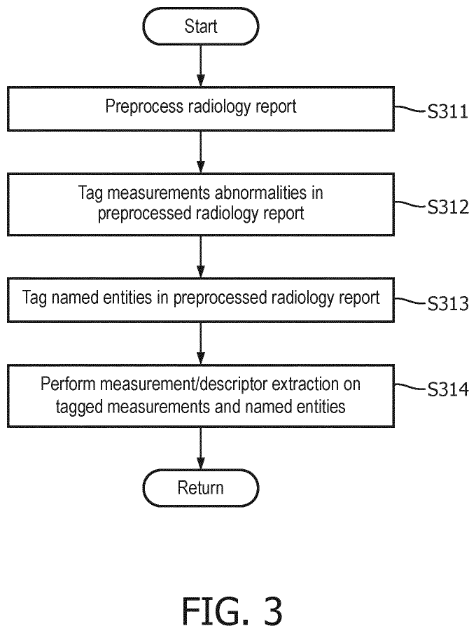

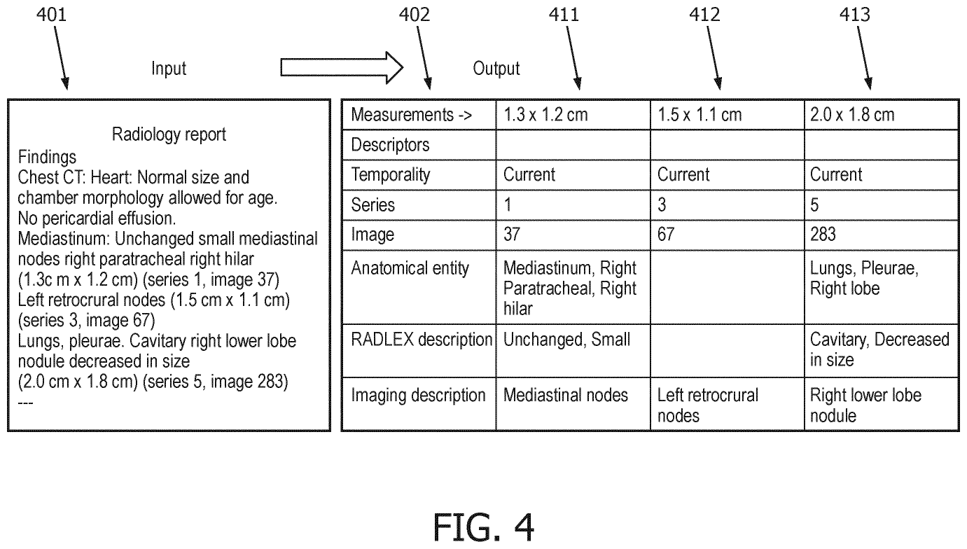

NLP module 144 is configured to execute one or more NLP algorithms using word embedding technology to extract measurements of abnormalities and corresponding descriptive text from the contents of the radiology report by processing and analyzing natural language data, as discussed below with reference to FIGS. 2 and 3. The NLP algorithm may split the radiology report into sections, such as a Findings section and an Impressions (Conclusion) section, entered by the user, and further split the sections into sentences. The NLP module 144 then evaluates the sentences, and extracts measurements of abnormalities observed in the current image, as well as descriptors associated with the measurements as entered by the user. The descriptors may include information such as temporality of a measurement (e.g., current or prior), a series number of the image for which the measurement is reported, an image number of the image for which the measurement is reported, an anatomical entity in which the associated abnormality is found, a RadLex® description of the status of the abnormality or other observation, an imaging description of the area being imaged, and a segment number of the organ being imaged, for example. Generally, RadLex® provides a comprehensive set of radiology terms for use in radiology reports to promote use of common language to communicate diagnostic results. The NLP module 144 causes the extracted information to be visually displayed on the display 124, an example of which is shown by NLP output 402 in FIG. 4. NLP is well known, and may include syntax and semantic analyses, for example, and deep learning for improving understanding by the NLP module 144 with the accumulation of data, as would be apparent to one skilled in the art.

The machine learning model module 145 is configured to measure reporting behavior of the user based on the output of the NLP module 144 according to a machine learning model or algorithm, as discussed below with reference to FIG. 2. The machine learning model module 145 may evaluate the use of descriptors extracted from the radiology report with regard to industry standard descriptors and/or descriptors commonly used by other radiologists in describing measurements of similar abnormalities. Based on the evaluation, the machine learning model module 145 may detect behavior patterns of the user within the radiology report, and detect practice variation of the user regarding use of standardized descriptors (e.g., industry standard descriptors and/or descriptors commonly used by users in other radiology reports). The machine learning model module 145 is may provide visibility into radiologist behavior patterns and practice variations when it comes to reporting descriptors of measurements, thereby improving content and consistency of the radiology report.

In an embodiment, the machine learning model module 145 is further configured to determine and analyze collective behavior patterns and practice variations of multiple users over time. For example, after a predetermined number of radiology reports are processed, the machine learning model module 145 may evaluate the use of descriptors extracted from these radiology reports with regard to industry standard descriptors and/or descriptors used by the participating users in describing measurements of similar abnormalities, according to the same machine learning model used for the individual evaluation or using a different machine learning model. The machine learning model module 145 is thus able to measure and provide collective behavior patterns and practice variations among the users. This information may be used as a reference for the users in preparing radiology reports going forward, and as a training tool for educating the users in order to increase awareness and to promote standardization and conformity of radiology reports among the users. The machine learning model module 145 is also able to provide information for determining how these behavior patterns and practice variations impact operational efficiency, reading workflow efficiency, and ultimately patient care.

In various embodiments, all or part of the processes provided by the NLP module 144 and/or the machine learning model module 145 may be implemented by an AI engine, for example. FIG. 2 is a flow diagram of a method of facilitating consistent use of descriptors by users describing medical images displayed on a display including a GUI, according to a representative embodiment. The method may be implemented by the system 100, discussed above, under control of the processor 120 executing instructions stored as the various software modules in the memory 140, for example.

Referring to FIG. 2, a medical image of a subject obtained during a current imaging exam is received and displayed with the GUI in block S211 for a particular study. The medical image may be received directly from a medical imaging device/modality (e.g., imaging device 160), such as an X-ray imaging device, a CT scan device, an MR imaging device, a PET scan device or an ultrasound imaging device, for example. Alternatively, or in addition, the medical image may be retrieved from a database (e.g., PACS database 112), for example, in which the medical image had been previously stored following the current imaging exam. The corresponding medical image may be displayed on a compatible display, such as a diagnostic viewer routinely used for reading radiological studies.

In block S212, contents of a radiology report are received from the radiologist via the GUI describing the medical image of the subject. The contents of the radiology report include measurements of one or more abnormalities (e.g., lesions, tumors) in the medical image and descriptive text associated with the measurements. The measurements and associated descriptive text may be included in the findings section and/or the impressions section of the radiology report, for example. The descriptive text includes descriptors associated with the measured abnormalities. The descriptors may be standardized descriptors, as discussed above, or may be improvised by the radiologist. The contents of the radiology report may also compare the medical image from the current imaging exam with one or more previous medical images from previous imaging exams (e.g., screenings, diagnostic exams). In this case, the contents would further include measurements of the abnormalities in the one or more previous medical images and associated descriptive text.

Generally, the findings section of the radiology report may include observations by the user about the medical images, and the impressions section may include conclusions and diagnoses of medical conditions or ailments determined by the user, as well as recommendations regarding follow-up treatment, testing, additional imaging and the like. The impressions section may also include comparisons of sizes of the abnormalities in the medical image with the one or more previous medical images and radiology reports, e.g., retrieved from the PACS database 112.

All or part of the contents of the radiology report may be dictated by the radiologist using a microphone of a user interface (e.g., interface 122), for example. Also, in an embodiment, receiving the contents of the radiology report may be interactive, where the GUI provides prompts for the radiologist to systematically measure and describe the abnormalities and other visual features of the medical image, and to enter findings and impressions. For example, the radiologist may be initially prompted to highlight apparent abnormalities in the medical image via the GUI, to perform and enter measurements of the highlighted abnormalities, and to enter corresponding descriptive text of the measurements. Alternatively, the abnormalities and/or measurements may be identified and performed automatically using well known image segmentation techniques, for example. In this case, the corresponding portions of the radiology report regarding abnormality identification and measurement may be populated automatically. The GUI may then prompt the user to enter the corresponding descriptive text with regard to the abnormalities and measurements.

Block S213 shows a process in which an NLP algorithm (NLP pipeline) is applied to the radiology report in order to extract measurements and corresponding descriptors in the descriptive text. The NLP algorithm parses the measurements and the descriptive text in the radiology report to identify numbers, key words and key phrases indicative of the measurements and the associated descriptors using well known NLP extraction techniques. The NLP extraction may be performed automatically, without explicit inputs from the radiologist who is reviewing the medical image. The measurements and corresponding descriptors may be displayed in tabular form, for example, as show in the example of FIG. 4. Generally, with regard to the NLP algorithm, relevant data from the contents can be extracted by applying domain-specific contextual embeddings for successful extraction of the measurements and descriptors from radiology reports. The NLP algorithm may be applied for the task of extraction of measurements and descriptors from the parsed sentences, and these clinical phrases may then be displayed to the radiologist.

FIG. 3 is a flow diagram of a method of applying an NPL algorithm for extracting feature measurements and corresponding descriptors from a radiology report, indicated in block S213 of FIG. 2, according to a representative embodiment. The method may be implemented by the system 100, discussed above, under control of the processor 120 executing instructions stored as the various software modules in the memory 140, such as NLP module 144, for example.

Referring to FIG. 3, preprocessing of the radiology report is performed in block S311 to provide preprocessed contents. The preprocessing includes performing a boundary protection algorithm to recognize sections and sentences of the radiology report. For example, the boundary protection algorithm may include a rules-based section splitter to split the radiology report into sections, such as the findings section and the impressions (conclusion) section, followed by sentence parsers that split the findings and impressions sections into sentences. That is, the sections and sentences are recognized, and the contents of the radiology report are split into the sections using regular expressions matched against a list of known section headers commonly used in radiology reports and segmented into sections. For example, lesion measurements and corresponding descriptive text are commonly recorded in the findings section, and analyzed in the impressions section. In an embodiment, all sections of the radiology report may be decoded to “utf-8” and split into sentences using the open source Python library Natural Language Toolkit (NLTK), for example. The preprocessing further includes lowercasing the descriptive text and removing punctuation.

In block S312, measurements of abnormalities (e.g., lesions, tumors) are tagged in the radiology report, along with temporalities associated with the abnormalities. Temporality is the determination of whether a measurement is a current measurement from the medical image, or a prior measurement from a previous medical image that has been included in the radiology report, e.g. for comparison or context. The measurements may be tagged using regular expression patterns and pre-defined rules. To detect the different measurements in each sentence, and to accurately tag the temporality the measurements, each sentence is divided into parts, which are created based on the number of measurements and their temporality, and complete descriptions of all elements of the measurements are captured. In particular, the sentence may be divided into two parts: a first part containing a current measurement, and a second part containing a prior measurement. Once the measurements are tagged, the sentences containing the tagged measurements may be output for tagging lesion entities, discussed below.

For example, measurements and associated temporalities may be tagged in the following illustrative sentence: “chest cavity mild increase in size of a left inferior lobe nodule, previously measuring 8×8 mm, now measuring 12×9 mm (7/288).” According to an embodiment, this sentence may be divided into a first sentence segment to record current measurements of the medical image, and a second sentence segment to record prior measurements of a prior medical image reference in the radiology report. In this example, the first sentence segment would be “chest cavity mild increase in size of a left inferior lobe nodule, now measuring 12×9 mm (7/288),” and the second sentence segment would be “previously measuring 8×8 mm.”

In block S313, named entities associated with the tagged measurements are tagged in the radiology report, including descriptors, such as series number and image number on which the measurement is reported, anatomical entity, RadLex® description, imaging description, and segment number of the organ involved in the imaging. In an embodiment, the named entities may be tagged using a conditional random fields (CRF) model to accommodate different writing styles, and linguistic and lexical variants of medical terms in radiology reports. Generally, a CRF model is a graphical model that discovers patterns in the descriptive text, given the context of a neighborhood, in order to capture many correlated features of inputs as well as sequential relationships among descriptors. The CRF model may be trained to achieve automatic named entity tagging for an anatomical entity, imaging observations, and descriptors associated with the feature measurements. For example, the named entity tagging may include tagging RadLex® description, including RadLex® sub-classes associated with the measurements. The CRF model receives the tagged measurements from block S312 and dictionary maps as input, and outputs label transition scores that help the radiologist explore and visualize relationships between the tagged descriptors associated with the tagged measurements. The label transition scores are conditional probabilities of possible next states given a current state of the CRF model and an observation sequence. In an embodiment, the CRF model may comprise a Python sklearn-crfsuite library with its model parameters to be used for tagging the named entities.

In block S314, rule-based extraction of the measurements and the associated descriptors is performed on the tagged measurements and the tagged named entities. The measurements and descriptors may be extracted using well known regular expression patterns and pre-defined rules. The extraction may focus on the seven types of descriptors that characterize a measurement in radiology: temporality, a series number of the image, an image number of the image, an anatomical entity in which the abnormality is found, a RadLex® (status) description, an imaging description of the area being imaged, and a segment number of the organ being imaged. The output from the extraction may be recorded as frames, in which each measurement is considered a target entity (primary entity) and all other entities (secondary entities) in the sentence segment containing the measurement are assumed to be related to the target entry as its descriptors. The secondary entities are labeled, where each label encodes the type of entity and the type of relation it has with the target entity. Accordingly, each measurement may be represented as a single frame object containing the numeric measure of the feature size and its associated descriptors as output from the NLP algorithm.

FIG. 4 shows a portion of an illustrative radiology report input to an NLP algorithm, and a corresponding output of the NLP algorithm, as provided by FIG. 3, for example, including extracted measurements and descriptors, according to a representative embodiment.

Referring to FIG. 4, input 401 includes contents of a radiology report as entered by the radiologist via a GUI, for example, as discussed above. In the depicted example, the contents include measurements of lesions in a current medical image with associated descriptive text. For purposes of illustration, the contents identify a first lesion as a mediastinal node measuring 1.3 cm×1.2 cm, a second lesion as a left retrocrural node measuring 1.5 cm×1.8 cm, and a third lesion as a right lower lobe nodule measuring 2.0 cm×1.8 cm.

Output 402 incudes the measurements and associated descriptors extracted from the contents of the radiology report shown in the input 401 by the NLP algorithm. In the depicted example, the output 402 is arranged such that the measurements define respective columns of descriptors of the lesions associated with the measurements. The descriptors include temporality, series number, image number, anatomical entity, RadLex® description, and imaging description. In an embodiment, the descriptors may further include a segment number of the organ being imaged, as mentioned above. For example, first column 411 of the output 402 identifies the measurement 1.3 cm×1.2 cm for the first lesion, and lists the associated temporality as “Current,” the series number as 1, the image number as 37, the anatomical entity as “Mediastinum, Right paratracheal, Right hilar,” the RadLex® description as “Unchanged, Small,” and the imaging description as “Mediastinal nodes.” Second column 412 identifies the measurement 1.5 cm×1.2 cm for the second lesion, and lists the associated temporality as “Current,” the series number as 3, the image number as 67, and the imaging description as “Left retrocrural nodes.” The anatomical entity and the RadLex® description are blank because the radiologist did not include this information in the radiology report for the second lesion. Third column 413 identifies the measurement 2.0 cm×1.8 cm for the third lesion, and lists the associated temporality as “Current,” the series number as 5, the image number as 283, the anatomical entity as “Lungs, Pleurae, Right lobe,” RadLex description as “Cavitary, Decreased in size,” and the imaging description as “Right lower lobe nodule.” The input 401 and/or the output 402 may be shown on the screen 126/GUI 128 of the display 124, for example.

Referring again to FIG. 2, once the NLP algorithm extracts measurements and corresponding descriptors from the radiology report, a machine learning model of reported measurements and associated descriptors is developed in block S214. The machine learning model evaluates the use of descriptors extracted from the radiology report with regard to standardized descriptors describing measurements of similar abnormalities. To this end, the machine learning model measures reporting behavior of the radiologist realistically and explores practice variation of the radiologist's use of descriptors relative to industry standards and/or reporting behavior of other radiologists.

For example, the machine learning model may detect behavior patterns of the radiologist with respect to completeness of recording measurements and the corresponding descriptors within the radiology report. That is, the machine learning model may detect the number and types of descriptors used by the radiologist in association with each of the measurements recorded in the radiology report, and identify internal variations and/or missing descriptors. Referring to FIG. 4, for example, the machine learning model may detect that the description of the second lesion lacks descriptors for the anatomical entity and the RadLex® description. With regard to practice variation, the machine learning model may compare descriptors extracted from the radiology report with a database of standard descriptors used in the medical field and/or with a database of similar descriptors built over time from radiologist reports by all radiologists using the same system.

In block 215, results of the machine learning model are optionally reported as feedback to the radiologist in order to improve the radiology report with regard to standardizing use of the descriptors and completeness of the radiology report. For example, the machine learning model may cause the behavior patterns and practice variations to be displayed to the radiologist to enable analysis of the quality of the radiologist report with respect to presence and use of the descriptors. In an embodiment, the machine learning model may even prompt the radiologist via the GUI to add missing descriptors to the radiology report, or to change descriptors to standard and/or more commonly used phraseology. The machine learning model thus is able to provide visibility into radiologist behavior patterns and practice variations when it comes to reporting descriptors of measurements, thereby improving content and consistency of the radiology report. The results of the machine learning model are also saved to a database of machine learning model results, collected from multiple radiology reports system wide.

In block S216, a collective machine learning model of all results regarding measurements and associated descriptors from the machine learning models for respective radiology reports is developed based on the machine learning model results collected from the multiple radiology reports. The collective machine learning model is used to determine and analyze the collective behavior patterns and practice variations of the contributing radiologists. In block S217, the collective machine learning model is used to measure collective behavior patterns and practice variations among the radiologists by comparing the descriptors, and to output a report with visualizations (displays) of use of the descriptors. For example, after processing 1,000 radiology reports from a group of different radiologists related to a specific indication/imaging modality pair, such as breast cancer and CT scan, the collective machine learning model of the results saved from the machine learning models for these radiology reports measures the behavior patterns and practice variations among the different radiologists when it comes to reporting standardized descriptors. Machine learning modelling radiologists' decisions to include certain descriptors is useful to understand their judgments. The collective machine learning model is also able to provide information for determining how the behavior patterns and practiced variations impact operational efficiency, reading workflow efficiency, and ultimately quality of patient care. The results of the collective machine learning model and visualizations are a ready reference available to the radiologists to enable future changes to standardize how descriptors are written.

The results of the collective machine learning model and visualizations are made available as a reference for radiologists applying descriptors in subsequent radiology reports or correcting previous radiology reports. For example, the results of the collective machine learning model make radiologists aware of various standardized descriptors to be included in radiology reports at the point of reading, ultimately creating more complete, clinically effective and definitive radiology reports. Therefore, it is ensured that the radiology reports include standardized descriptors consistent with industry standards and/or other multiple radiology reports. The results and visualizations may also be used as a reference and a training tool for the radiologists in order to increase awareness and to promote standardization and conformity of radiology reports among the group of radiologists. Also, results and visualizations provide information for determining how these behavior patterns and practice variations impact operational efficiency, reading workflow efficiency, and ultimately quality of patient care.

Generally, the collective behavior patterns and practice variations with respect to completeness of reported measurements and associated standardized descriptors help to unify reporting of measurements among many radiologists, improve upon radiology reports and better diagnostic certainty. That is, the output of the collective machine learning model shows radiologist practice variation, for example, with regard to missed descriptors that can be corrected at an individual radiologist level or at an aggregate level. This supports better reported diagnoses toward greater definitiveness and understandability. Missed descriptors not only indicate variation in practice, but also may have critical implications for patient care resulting from misunderstood criticality of lessons and other abnormalities, which may lead to delayed or otherwise inadequate treatment.

In accordance with various embodiments of the present disclosure, the methods described herein may be implemented using a hardware computer system that executes software programs stored on non-transitory storage mediums. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Although facilitating consistent use of descriptors related to measurements in the preparation of radiology reports on medical images has been described with reference to exemplary embodiments, it is understood that the words that have been used are words of description and illustration, rather than words of limitation. Changes may be made within the purview of the appended claims, as presently stated and as amended, without departing from the scope and spirit of interventional procedure optimization in its aspects. Although facilitating the consistent use of descriptors in the preparation of radiology reports has been described with reference to particular means, materials and embodiments, facilitating the reading of medical images is not intended to be limited to the particulars disclosed; rather facilitating the consistent use of descriptors in the preparation of radiology reports extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A method of facilitating consistent use of descriptors describing medical images, the method comprising:
receiving contents of a radiology report from a user via a graphical user interface (GUI) describing a medical image of a subject, the contents comprising a measurement of at least one abnormality appearing in the medical image and descriptive text corresponding to the measurement, the descriptive text comprising at least one descriptor of the measurement;
extracting the measurement and the at least one descriptor from the contents of the radiology report using a natural language processing (NLP) algorithm;
developing a machine learning model including the measurement and the at least one descriptor, wherein the machine learning model determines at least one of a behavior pattern or a practice variation of the user with respect to use of the at least one descriptor relative to industry standards and/or reporting behavior of additional users;
developing a collective machine learning model of results of the developed machine learning model regarding the behavior patterns and/or the practice variation of the user and results of developed machine learning models regarding behavior patterns and/or practice variations of the additional users based on additional radiology reports from the additional users; and
measuring collective behavior patterns and collective practice variations using collective machine learning model, the measured collective behavior patterns and the collective practice variations being made available as reference to the user.

2. The method of claim 1, wherein extracting the measurement and the corresponding descriptive text from the contents of the radiology report using an NLP algorithm comprises:
preprocessing of the radiology report to provide preprocessed contents;
tagging the measurement in the preprocessed contents;
tagging a named entity corresponding to the tagged measurement in the preprocessed contents; and
performing rule-based extraction of the measurement and the at least one descriptor on the tagged measurement and the tagged named entity.

3. The method of claim 2, wherein the preprocessing comprises:
splitting the radiology report into sections;
parsing sections into sentences; and
lowercasing the descriptive text and removing punctuation.

4. The method of claim 2, wherein the measurement is tagged using regular expression patterns and pre-defined rules.

5. The method of claim 4, wherein tagging the measurement comprises:
dividing each sentence of the radiology report a first part containing the measurement of the at least one abnormality, and a second part containing a prior measurement.

6. The method of claim 2, wherein the named entity is tagged using a conditional random fields (CRF) model.

7. The method of claim 2, wherein performing rule-based extraction comprises:
recording an output of the rule-based extraction as frames, in which the measurement is considered a target entity and all other entities are assumed to be related to the target entry as the at least one descriptor;
labeling the other entities, wherein each label encodes a type of entity and a type of relation the entity has with the target entity; and
representing the measurement as a single frame object containing the measurement and the at least one descriptor.

8. The method of claim 1, wherein the at least one descriptor comprises one or more of temporality, a series number of the medical image, an image number of the medical image, an anatomical entity in which the at least one abnormality is found, a status description of a status of the abnormality; an imaging description of an area being imaged, and a segment number of the organ being imaged.

9. The method of claim 1, wherein the contents of the radiology report are received from the user through dictation.

10. A system for facilitating consistent use of descriptors describing medical images, the system comprising:
a processor;
a graphical user interface (GUI) enabling a user to interface with the processor; and
a non-transitory memory storing instructions that, when executed by the processor, cause the processor to:
receive contents of a radiology report from the user via the GUI describing a medical image of a subject, the contents comprising a measurement of at least one abnormality appearing in the medical image and descriptive text corresponding to the measurement, the descriptive text comprising at least one descriptor of the measurement;
extract the measurement and the at least one descriptor from the contents of the radiology report using a natural language processing (NLP) algorithm;
develop a machine learning model including the measurement and the at least one descriptor, wherein the machine learning model determines at least one of a behavior pattern or a practice variation of the user with respect to use of the at least one descriptor relative to industry standards and/or reporting behavior of additional users;
develop a collective machine learning model of results of the developed machine learning model regarding the behavior patterns and/or the practice variation of the user and results of developed machine learning models regarding behavior patterns and/or practice variations of the additional users based on additional radiology reports from the additional users; and
measure collective behavior patterns and collective practice variations using collective machine learning model, the measured collective behavior patterns and the collective practice variations being made available as reference to the user.

11. The system of claim 10, wherein the instructions cause the processor to extract the measurement and the corresponding descriptive text from the contents of the radiology report using an NLP algorithm by:
preprocessing the radiology report to provide preprocessed contents;
tagging the measurement in the preprocessed contents;
tagging a named entity corresponding to the tagged measurement in the preprocessed contents; and
performing rule-based extraction of the measurement and the at least one descriptor on the tagged measurement and the tagged named entity.

12. The system of claim 11, wherein the instructions cause the processor to preprocess the radiology report by:
splitting the radiology report into sections;
parsing sections into sentences; and
lowercasing the descriptive text and removing punctuation.

13. The system of claim 11, wherein the instructions cause the processor tag the measurement using regular expression patterns and pre-defined rules.

14. The system of claim 13, wherein the instructions cause the processor tag the measurement by:
dividing each sentence of the radiology report a first part containing the measurement of the at least one abnormality, and a second part containing a prior measurement.

15. The system of claim 11, wherein the instructions cause the processor to tag the named entity using a conditional random fields (CRF) model.

16. The system of claim 11, wherein the instructions cause the processor to perform rule-based extraction by:
recording an output of the rule-based extraction as frames, in which the measurement is considered a target entity and all other entities are assumed to be related to the target entry as the at least one descriptor;
labeling the other entities, wherein each label encodes a type of entity and a type of relation the entity has with the target entity; and
representing the measurement as a single frame object containing the measurement and the at least one descriptor.

17. The system of claim 10, wherein the at least one descriptor comprises one or more of temporality, a series number of the medical image, an image number of the medical image, an anatomical entity in which the at least one abnormality is found, a status description of a status of the abnormality; an imaging description of an area being imaged, and a segment number of the organ being imaged.

18. The system of claim 10, wherein the contents of the radiology report are received from the user via the GUI by dictation.
